(19) United States
(12) Patent Application Publication
(10) Pub. No.: US 2009/0209849 A1
(43) Pub. Date: Aug. 20, 2009

(54) Medical Device Placement and Monitoring System Utilizing Radio Frequency Identification

(76) Inventors: Philip Stephen Rowe, Port Saint Lucie, FL (US); Jimmie Hanson Spraker, JR., Pleasanton, CA (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/428,664

(22) Filed: Apr. 23, 2009

Related U.S. Application Data
(63) Continuation-in-part of application No. 11/799,664, filed on May 2, 2007.

(51) Int. Cl. A61B 5/05; G08B 5/22

(52) U.S. Cl. 600/424; 340/825.49

(57) ABSTRACT

A medical device placement and monitoring system is provided that includes numerous circuits that can be used alone or in combination. The circuits include a feedback initiator, a feedback receiver, and a clinician notifying device. By various means in the provided circuits, the feedback initiator provides information about the location of a radio frequency identification tag secured to a medical device inserted within a patient. This information or data is received and analyzed by the feedback receiver that monitors the circuit, which then transmits an output to the clinician notifying device to alert or advise the attending clinician of this information. The data that is output supplies information about the location of the radio frequency identification tag to the clinician, thereby assisting the clinician in placement of a variety of medical devices including nasogastric or endotracheal tubes, as well as in monitoring proper device placement after initial insertion.

### CROSS REFERENCE TO RELATED APPLICATIONS

This application is a continuation-in-part of Ser. No. 11/799,664, filed with the U.S. Patent and Trademark Office on May 2, 2007, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention relates generally to a medical device that is used to correctly place and monitor either a nasogastric tube during insertion procedures into the stomach or an endotracheal tube for patient airway intubations into the lungs. This medical device serves as a placement and monitoring system configured to assure proper placement—either into the stomach for a nasogastric tube or into the lungs for an endotracheal tube—and monitoring thereafter of either the nasogastric or endotracheal tube. The system is composed of various circuits that may provide feedback in real time for pH/continuity levels, CO2 levels, auscultatory output, temperature levels, and the like. The system may also comprise air pressure sensors, indicator lights, meters, and/or speakers. In one embodiment, the present inventive system may be used in combination with a nasogastric tube that is inserted through a patient's nose and down through the alimentary canal into the stomach. In a second embodiment, the present inventive system may be used in combination with an endotracheal tube that is inserted directly into the lung(s) of a patient to provide airflow. When the organ of interest is the stomach, the nasogastric tube may be used to deliver hydration, nutrition, and medications to the patient. An endotracheal tube may be used to provide an airway for a patient and for maintenance of that airway through mechanical ventilation.

2. Background Art

The use of a nasogastric tube, a plastic tubular conduit inserted through the nose into the throat down through the alimentary canal and into the stomach, is an important standard therapeutic technique. The nasogastric tube is commonly used to introduce materials such as nutrition, hydration, or medications into the stomach. It is also used to decompress the stomach to prevent vomiting after major surgery and for removing material from the body, such as an accidentally ingested poison, an overdose of drugs, or other toxins that have built up in the stomach, perhaps due to surgery, or gastrointestinal blockages. It is widely recognized that enteral nutrition provided by a nasogastric tube, or a feeding tube as it is sometimes referred to, is preferred to parenteral nutrition, as nasogastric tubes present less complications.

Where the lung is the organ of interest, any suction tube can be used for the purpose of suctioning or draining fluid. One common example is with a patient who may have pneumonia.

Although nasogastric insertions or endotracheal intubations are widely used, standard techniques in hospitals, long term care facilities, hospices, and in-home health care delivery, it continues to be challenging for health care providers to assure proper placement of either a nasogastric or endotracheal tube. Great care and attention must be used to assure the tube is correctly placed.

The organ of interest for a nasogastric tube placement is the stomach. The inadvertent misplacement of nasogastric tubes by health care providers has caused numerous health problems for patients. Nasogastric tubes are often inadvertently placed into the tracheobronchial tree or pleural space, when the organ of interest for placement is the stomach. Aspiration by proxy is a serious consequence of inadvertent misplacement of a nasogastric tube. This occurs when food or medicine is introduced by a nasogastric tube directly into the lungs, which can cause aspirated pneumonia with its associated increased incidence of morbidity and mortality. Improper nasogastric tube placement has led to laryngeal injuries due to placement in the trachea and distal airways, when the organ of interest was the stomach. Other related issues include hypersalivation, depressed cough reflex, and pharyngeal abnormalities.

Determining correct placement of either a nasogastric tube or an endotracheal tube is vital. Complications from improper tube positioning often result in extended hospital stays, or in some instances, may result in death.

The importance of proper insertion procedures and the difficulty of assuring correct placement have led to the development of numerous confirmation techniques that are used either individually or in combination with others to assess proper placement of a nasogastric tube. Some of these include capnometry, capnography, auscultation, and endoscopic fluoroscopic techniques. Similar confirmation techniques have been designed for endotracheal tube intubations.

Radiographic confirmation of the location of the distal end of the tube, however, is the most reliable confirmation technique. Even when an experienced clinician blindly places a nasogastric tube or the like, placement must then be verified by radiographic confirmation. A standard tube has a radio-opaque marker or strip at the distal end, so the position can be verified by X-ray studies of the chest/abdomen. If the X-ray cannot confirm the position, an alternative technique known as fluoroscopy can be used to confirm the distal end location. Radiographic assistance can also be used during the insertion of the tube.

While radiographic confirmation does assure correct placement of a nasogastric tube, the patient is exposed to radiation, and the cost of radiographic confirmation is costly and radiographic confirmation is difficult or impossible to provide in some situations, such as within in-home health care environments. Additionally, some patients that require nasogastric tubes have multiple pieces of life support equipment. Therefore, a substantial amount of time, effort, and hospital staff are required to move, position, and manage these patients while performing the radiographic confirmation.

It would be advantageous to provide a system that can safely and correctly place and monitor a nasogastric tube, an endotracheal tube, and the like while incurring a significantly lower cost than the traditional radiographic confirmation. The need for specialized staff to perform the various placement and monitoring techniques could also be greatly reduced. A method provided through a system that can offer continuous verification of the position of the distal end of the tube both during the insertion of a nasogastric tube or the intubation of an endotracheal tube as well as during the entire intubation period would be advantageous for both the patient and the attending hospital staff.

A traditionally used bedside technique to evaluate the placement of a nasogastric tube is auscultation of air insufflated through the tube. In this method a trained technician using a stethoscope above the stomach rapidly fills the tube with a bolus of air and determines whether the sound generated by the air injected into the tube is from the gastrointestinal system, from the respiratory system, or other location. This is a very economical test method, but the amount of training and clinical experience required is substantial. Additionally, this method is very time consuming, as the trained clinician attempts to correctly differentiate the sounds to determine the location of the distal end of the nasogastric tube. Furthermore, this method does not deliver a high degree of accuracy.

Another placement evaluation method involves aspiration of fluid from the tube, followed by pH testing of the aspirate. By using pH paper, the acidity of the fluid can be determined. An acidic pH of approximately lower than 5 may indicate the correct placement of a nasogastric tube in the stomach, while an aspirate of pH 6 or greater indicates that the nasogastric tube may be inadvertently positioned in the respiratory system. One problem associated with this method of using the aspirate of the tube is the tendency for small-bore tubes to collapse when suction is applied. Additionally, aspirating fluid requires a significant investment of time and effort by the trained clinician. Also, it is difficult to obtain an aspirate from the tube in dehydrated patients or in certain areas of the stomach where there may be no pool of fluid of sufficient volume to aspirate. It would be advantageous to have a device that decreased the amount of time spent by hospital personnel to aspirate fluid and to test the pH of the fluid every time a pH value was desired.

Even after a successful initial placement of a nasogastric tube is confirmed, the patient faces an ongoing risk. This is because over time the distal end of the tube can become mal-positioned, moving from its original location. For example, this may occur due to patient movement or the patient may dislodge the tube because it is uncomfortable. Commonly hospital policies recommend frequent and ongoing placement confirmation, for example, before every feeding or at least every six hours. Obviously a great deal of radiation exposure would be received if this confirmation were done by radiology, as well as being financially costly. It would be advantageous to have a device for continual monitoring of the location without the expense and the radiation exposure of repeated X-rays.

Accordingly, there is an established need for a timesaving, economical, nasogastric or endotracheal tube placement and monitoring system that will guide a clinician during the placement of the nasogastric or endotracheal tube, so that proper placement is achieved into the organ of interest, such as the stomach for a nasogastric tube or the lungs for an endotracheal tube, while additionally providing proper monitoring during the entire intubation period and eliminating the need for radiological confirmation as the main procedure to assure proper placement. In this manner, the present invention provides and establishes a standardization of procedure upon which all caregivers can depend.

### BRIEF SUMMARY OF THE INVENTION

In accordance with one embodiment, a placement and monitoring system for use by a clinician during insertion of a medical device within a patient, comprising a radio frequency identification tag secured to the medical device, the radio frequency identification tag comprising at least one feedback initiator configured to provide information concerning the location of the radio frequency identification tag within the patient, at least one feedback receiver configured to receive the information from the at least one feedback initiator, and at least one notifying device configured to present the information concerning the location of the radio frequency identification tag to the clinician, wherein the information concerning the location of the radio frequency identification tag within the patient is communicated from the at least one feedback initiator to the at least one feedback receiver via transmission of a radio frequency signal.

The present invention is directed to an economical, time-saving, efficient, nasogastric or endotracheal tube placement and monitoring system that is capable of assisting clinicians with proper tube placement during the procedure and also of advising the clinician during the entire placement procedure, by continually monitoring and providing information regarding the location of the distal end of the tube. This system may comprise numerous circuits that can be used alone or in combination, wherein the system may be used with a flexible nasogastric tube, an endotracheal tube, catheter, or any other known medical device. The circuits may include a feedback initiator, a feedback receiver, and a clinician notifying device. By various means in the provided circuits, the feedback initiator provides information about the location of the distal end of the tube at all times during placement. This information or data is received by the feedback receiver that monitors the circuit. The feedback receiver receives the information about the location of the distal end of the tube from the feedback initiator, and transmits an output to a clinician notifying device to alert or advise the attending clinician of this information. In a preferred embodiment of provided circuits, a radio frequency signal is used to connect the feedback initiator disposed on the distal tip of a nasogastric or endotracheal tube to the feedback receiver disposed within a control panel or other graphical user interface device.

In the provided system and method, the feedback initiator may be any of a variety of components including but not limited to an air pressure sensor, a CO2 sensor, a pH continuity circuit, a built-in electronic stethoscope within the system itself, and the like. In one embodiment of the present invention, the system may inject a small amount of air, known as air insufflation, that will bubble air out the distal end of the tube in order to clear fluid that is clogging the tube and further provides a secondary level of confirmation that the tube is located in the stomach by placing a hand held stethoscope over the patient's stomach to ascertain placement. The feedback receiver may be any of a variety of components including but not limited to a pH continuity meter, an air pressure sensor, an electronic stethoscope, a CO2 sensor, and the like. The output clinician notifying system may also be any of a variety of sub-systems such as a graphical user interface (GUI) having an LCD touch panel display, warning indicator lights, an alarm, system feedback and status, control input and output, a “reset” feature, and the like. Other features may include a battery level condition indicator that may display current voltage as well as energy status. The graphical user interface may display the current level of software and firmware and provide an additional feature to accommodate “green” concerns of governing bodies. In this manner, all features and functions of the system may be displayed on a graphical user interface. The data that is output by this system to the control panel or graphical user interface supplies information about the location of the tube's distal end to the clinician and thereby assists the clinician in placement of the nasogastric or endotracheal tube and in monitoring tube position after placement has been achieved into the organ of interest.

An object of the present invention is to provide a nasogastric or endotracheal tube placement and monitoring system that confirms tube placement without the use of radio graphic confirmation.

Another object of the present invention is to provide a nasogastric or endotracheal tube placement and monitoring system that transmits data or information regarding the placement of a distal tip of a medical device within a patient via a wireless radio frequency signal.

An additional object of the present invention is to provide a nasogastric or endotracheal tube placement and monitoring system that eliminates the patient's exposure to radiation through radiographic confirmation.

A further object of the present invention is to provide a nasogastric or endotracheal tube placement and monitoring system that offers a significantly lower cost than the cost of the traditional radiographic confirmation.

An additional object of the present invention is to provide a nasogastric or endotracheal tube placement and monitoring system that reduces patient health risks associated with improper placement.

Another object of the present invention is to provide a nasogastric or endotracheal tube placement and monitoring system that is configured to continuously monitor the location of the distal end of the tube.

A further object of the present invention is to provide a nasogastric or endotracheal tube placement and monitoring system that improves staff efficiency in terms of tube placement procedures.

These and other objects, features, and advantages of the present invention will become more readily apparent from the attached figures and the detailed description of the preferred embodiments, which follow.

### BRIEF DESCRIPTION OF THE DRAWINGS

The preferred embodiments of the invention will hereinafter be described in conjunction with the appended figures provided to illustrate and not to limit the invention, where like designations denote like elements, and in which:

FIG. 1 is a perspective view showing an overview of an embodiment of a nasogastric tube placement and monitoring system of the present invention in a typical application, illustrating placement in the stomach.

FIG. 2 is a side view of a generalized schematic diagram of an embodiment of a radio frequency identification tag of the present invention.

FIG. 3 is a front view of an embodiment of a graphical user interface of the present invention, wherein the graphical user interface is displaying real-time information regarding nasogastric tube placement and monitoring.

FIG. 4 is a front view of an embodiment of a graphical user interface of the present invention, wherein the graphical user interface is displaying real-time information regarding endotracheal tube placement and monitoring

FIG. 5 is a perspective diagrammatic view showing the pH monitoring circuit/continuity circuit of a preferred embodiment of the nasogastric or endotracheal tube placement and monitoring system of the present invention as implemented with a distal pH sensor and/or continuity conductor.

FIG. 6 is a diagrammatic flowchart showing the pH monitoring circuit/continuity circuit of a preferred embodiment of the nasogastric or endotracheal tube placement and monitoring system of the present invention.

FIG. 7 is a perspective diagrammatic view showing an audio monitoring circuit of an embodiment of the nasogastric or endotracheal tube placement and monitoring system of the present invention illustrating an audio monitoring circuit as implemented with a proximal acoustic feedback receiver, such as a microphone.

FIG. 8 is a diagrammatic flowchart showing an audio monitoring circuit of the an embodiment of the nasogastric or endotracheal tube placement and monitoring system of the present invention.

FIG. 9 is a perspective diagrammatic view showing air pressure sensor circuit of an embodiment of the nasogastric or endotracheal tube placement and monitoring system of the present invention illustrating an air pressure circuit as implemented with a proximal air pressure sensor.

FIG. 10 is a diagrammatic flowchart showing the air pressure sensor circuit of an embodiment of the nasogastric or endotracheal tube placement and monitoring system of the present invention.

FIG. 11 is a front diagrammatic view showing one embodiment of a control panel of the nasogastric or endotracheal tube placement and monitoring system of the present invention, illustrating the use of a combination of the circuits of the present invention, as implemented with a combination of feedback initiators, feedback receivers, and notifying devices.

FIG. 12 is a diagrammatic flowchart showing the stomach monitoring circuit of an embodiment of the nasogastric or endotracheal tube placement and monitoring system of the present invention.

FIG. 13 is a diagrammatic flowchart showing the lung monitoring circuit of an embodiment of the nasogastric or endotracheal tube placement and monitoring system of the present invention.

FIG. 14 is a diagrammatic flowchart showing the location monitoring circuit of an embodiment of the nasogastric or endotracheal tube placement and monitoring system of the present invention.

Like reference numerals refer to like parts throughout the several views of the figures.

### DETAILED DESCRIPTION OF THE INVENTION

Although the following detailed description contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention.

Shown throughout the figures, the present invention is directed toward a nasogastric or endotracheal tube placement and monitoring system that provides information about the location of the distal end of the nasogastric or endotracheal tube. The nasogastric or endotracheal tube placement and monitoring system of the present invention is not only capable of assisting clinicians during placement procedures but also capable of allowing clinicians to determine the location at all times after placement and continually thereafter during the hours or days that such a medical system is being utilized. A variety of sub-systems or circuits are provided, which may be used individually or in any combination to provide the clinician with optimum information concerning the location of the distal end of the tube or other medical device.

While the various systems or circuits provided are described or identified by the term “circuit”, which may refer to a path of an electric current, the term is herein used more broadly to also encompass a path of other data or information, such as, for example, sound waves.

Additionally, while the majority of illustrated embodiments and figures refer to nasogastric embodiments of the present inventive disclosure, the scope of the present invention further includes but is not limited to endotracheal applications as well as any other medical tube, catheter, or medical device applications and procedures known within the art. Nasogastric and endotracheal applications are disclosed merely for ease of explanation and illustration, whereas such disclosures may be applied to all other medical devices and procedures known within the art. The endotracheal application of the present inventive system may provide for endotracheal tube placement into the lungs to provide airway management for the patient. It also provides a manner in which to sample air and continuously monitor “live” pH readings of the patient from a monitoring perspective.

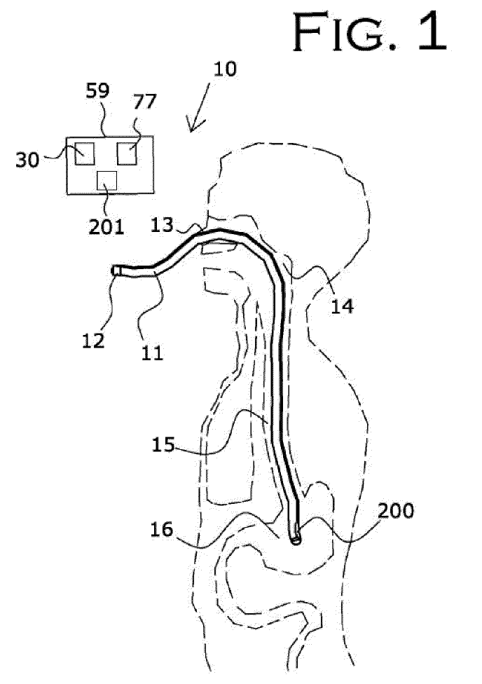

Referring now to FIG. 1, an embodiment of a nasogastric tube placement and monitoring system, shown generally as reference number 10, is illustrated as an overview to depict preferred embodiments of the present invention. As shown, the nasogastric or endotracheal tube placement and monitoring system 10 of the present invention generally includes an radio frequency identification (RFID) tag 200 containing a feedback initiator 20 (not shown), and a feedback receiver 30 and a clinician notifying device 77 preferably disposed within a user interface such as control panel 59. The placement and monitoring system of the present invention may be used in combination with a wide variety of medical device including but not limited to a flexible tube 11, such as a nasogastric (see FIG. 1) or endotracheal tube.

The standard nasogastric flexible tube known in the art, as well as a nasogastric flexible tube 11 of the present invention, has a distal end that is inserted into the patient, a proximal end that remains outside the patient, and an extended midsection to provide sufficient length for the intubation procedure. Both the standard nasogastric tube and a nasogastric flexible tube 11 of the present invention are adapted and sized for insertion into a nasal passage 13 of a patient. The flexible tube 11 can be of any conventionally available sizes or materials, and includes single, double, and triple lumen tubes, as are known in the art. If desired for the intended procedure, the proximal end of flexible tube 11 may be configured with a standard adapter 12 for connection to standard medical equipment or supplies, for example ready-to-hang, enteral nutrition containers.

The nasogastric flexible tube 11 may be used in combination with a feedback initiator 20 of the present invention, wherein the feedback initiator 20 is disposed within an RFID tag 200 on the distal end of the flexible tube 11 in most provided circuits.

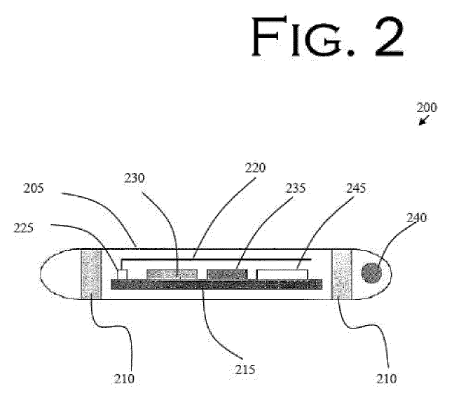

In such embodiments of the circuits provided, the flexible tube 11 is configured with an RFID tag 200 mounted on, incorporated into, or connected to the distal end of flexible tube 11. Within the scope of the present invention, the RFID tag 200 may be associated with any medical device, including but not limited to the distal end of a nasogastric or endotracheal tube, which provides for numerous features not found in RFID devices on the market today. The RFID tag 200 may further comprise a feedback initiator 20 and a radio frequency (RF) transmitter 225 (see FIG. 2) for communicating data from the distal end of the flexible tube 11 or other medical device.

Such a novel RFID tag 200 used within the scope of the present invention and as described above may collect and allow for live or “real time” data monitoring including but not limited to temperature, CO2 levels, and pH values immediately adjacent the RFID tag 200 inserted within a patient's body.

FIG. 1 depicts an RFID tag 200 disposed at the distal end of a nasogastric tube as embodied by flexible tube 11. Due to the radio frequency communication between the feedback initiator 20 disposed within the RFID tag 200 and an external feedback receiver 30, the need for any restrictive wiring configuration is eliminated. Such wireless radio frequency communication provided by the system of the present invention allows for a much greater freedom of movement between the feedback initiator 20 and the independent external feedback receiver 30.

The scope of the present inventive system is not limited to placement and monitoring of a nasogastric tube, but may further include placement and monitoring of an endotracheal tube as well as all other tubes or catheters to be inserted into a patient's body. The radio frequency embodiment disclosed herein provides for a system that is easily incorporated onto any insertable tube, catheter or any other medical device, wherein the inventive system wirelessly provides real-time time data or information regarding the internal bodily environment immediately adjacent the feedback initiator 20 disposed within the RFID tag 200.

As shown in FIG. 1, the nasogastric or endotracheal tube placement and monitoring system 10 of the present invention may generally include a feedback initiator 20 disposed within an RFID tag 200, a radio frequency reader and detection system 201 for receiving the radio frequency signal from the RFID tag 200, a feedback receiver 30 for receiving the collected data, a clinician notifying device 77, and, preferably, a user interface or control panel 59.

FIG. 2 illustrates a generalized schematic diagram of one embodiment of an RFID tag 200 of the present invention. As shown, an RFID tag 200 of the present invention may comprise a capsule 205, two or more silver chloride contacts or semi-permeable membranes 210, a temperature sensor 240, a microcontroller 215, an antenna 220, an RF transmitter 225, an analog to digital converter 230, a Hi “Z” Op-Amp 235, a power supply 245, and the like. Information or data transmissions from the RFID tag 200 may be read by an external RFID reader and detection system 201 (see FIG. 1). The RFID tag 200 may operate on a specific frequency as the base carrier and transmit information or data that may be accurately acquired, read, and recorded by the external RFID reader and detection system 201.

Such information or data may be stored in a non-volatile memory within the RFID tag 200 and may be read at anytime by an external RFID reader and detection system 201. When the RFID tag 200 is within the RFID reader's range, positioning and placement of the RFID tag 200 may be discerned, an event may be triggered when the RFID tag 200 is in position, and an alarm may sound if the RFID tag 200 moves either to an undesired bodily position or outside the range of the external RFID reader and detection system 201. The disclosed functionality may further be influenced by the size and shape of the receiving antenna.

The RFID tag 200 may comprise one or more unique design embodiments to assist in providing real-time placement and monitoring capabilities. In one embodiment, the capsule 205 may comprise a plastic tube within which the electronic components of the RFID tag 200 may be disposed. An additional embodiment may comprise a glass tube in which the electronic components of the RFID tag 200 are disposed. The use of a plastic tube capsule 205 meets the FDA's biocompatibility requirements and may allow for easier mounting of the silver chloride contacts or semi-permeable membranes 210 thereon, as compared to a glass capsule 205 embodiment of the present invention.

The design of the antenna 220 within the RFID tag 200 is also distinct from traditional RFID technology. Conventional RFID devices have the greatest signal strength when measured from either end of the device. In one embodiment of the present invention, the antenna 220 may be located within the RFID tag 200 and be positioned to allow the greatest signal strength to be measured along the length of the device. In this manner, the RFID tag 200 is perpendicular to the RFID reader and detection system 201 and allows for the detection of an RFID tag 200 that is disposed on a medical device within the human body.

The microcontroller 215 of the present invention may be any microcontroller known within the art that allows for data acquisition, processing, and storage of the data received in a non-volatile memory. It is essential that the power usage of the microcontroller 215 be as minimal as possible due to the ultra-low power supply current that will be available as derived from the radio frequency energy signal that may be received from the external RFID reader and detection system 201.

Data may be transmitted from the RFID tag 200 to the external RFID reader and detection system 201 via a radio frequency (RF) transmitter 225 contained within the RFID tag 200. The RF transmitter 225 may reside on the same printed circuit board as the microcontroller 215. In a preferred embodiment, the RF transmitter 225 may have sufficient strength to relay any collected information or data to the external RFID reader and detection system 201 over a distance of no less than 5 centimeters with a preferred target range of 10 centimeters. The power supply 245 of the RFID tag 200 may be powered by a radio frequency energy source provided by the external RFID reader and detection system 201. The power supply 245 must have sufficient power (e.g. volts and current) to supply all of the active electronics that are present within the RFID tag 200 of the present invention. Additionally, the power supply 245 may be enabled by the conductivity between the two or more silver chloride contacts or semi-permeable membranes 210, as described herein.

In use, the RFID tag 200 may remain static until it is stimulated by an external radio frequency energy source that may be provided by the external RFID reader and detection system 201. Upon receipt of the radio frequency signal from an RFID reader and detection system 201, the RFID tag 200 becomes activated via the received radio frequency energy and begins taking samples in “real-time” of information or data including but not limited to temperature, CO2 levels, pH value, and the like. Such information or data readings may then be stored in a non-volatile memory disposed within the RFID tag 200. In one example, the position of the distal end of a nasogastric tube having an RFID tag 200 of the present invention may be verified by the display of a pH value, such as that found within the human stomach. The pH value may be communicated as a data reading to the microcontroller 215. The circuit of the RFID tag 200 may be enabled when the conductivity between the two or more silver chloride contacts or semi-permeable membranes 210 is within a predetermined pH value range. Likewise, continuity may be achieved by monitoring the conductance between the two or more silver chloride contacts or semi-permeable membranes 210, wherein the signal is amplified and processed via the Hi “Z” op-amps 235, converted by the analog to digital converter 230, and communicated to the microcontroller 215 for data processing and formatting. If the microcontroller 215 detects that continuity or hydrogen ions are within the specified range, indicating that the RFID tag 200 is in conductive media (using pH as an indicator of conductivity), then the microcontroller 215 will enable radio frequency transmissions from the RFID tag 200.

The predetermined data ranges, such as pH value range, temperature range, CO2 range, and the like (i.e. proper continuity) may be preprogrammed into the microcontroller 215. In this manner, an RFID tag 200 of a nasogastric tube embodiment may be preprogrammed differently than an RFID tag 200 of an endotracheal tube embodiment of the present invention due to the different pH values, temperature ranges, or CO2 ranges found in their respective target organs. Any variance from a predetermined and preprogrammed data range such as pH value range, temperature range (e.g. proper continuity), or CO2 range stored within the microcontroller 215 may allow for signaling the misplacement of the distal end of the nasogastric or endotracheal tube, respectively.

In the nasogastric or endotracheal tube placement and monitoring system of the present invention, the control panel 59 is preferably configured to be placed near a patient's bed at an easily readable and accessible location. The control panel 59 serves as a user interface, allowing the clinician to easily and conveniently manually initiate the one or more circuits to be used in the nasogastric or endotracheal tube placement and monitoring system of the present invention, as well as providing the feedback from the distal end of the nasogastric or endotracheal tube to the clinician in an accessible and readily available manner.

Control panel 59 integrates the various elements of the one or more (preferably multiple) circuits used for the nasogastric or endotracheal tube placement and monitoring system, providing a housing for the required monitoring electronics and for the clinician notifying devices such as displays or speakers, as utilized by the one or more circuits. Control panel 59 is configured with the necessary electronics for the circuits and systems provided in the nasogastric or endotracheal tube placement and monitoring system of the present invention. Preferably, control panel 59 may be incorporated into an aesthetically pleasing cabinet or other enclosure that is easy to clean, to maintain, and to move to the location of use.

Feedback receiver 30 is preferably disposed in control panel 59. Feedback receiver 30, in one embodiment of provided circuits, is in communication with the feedback initiator 20 via an RF signal transmitted there between. RF communication allows flexible tube 11 and/or RFID tag 200 to be disposable if desired, while feedback receiver 30 may preferably be reusable such as when disposed in control panel 59 or, optionally, disposable.

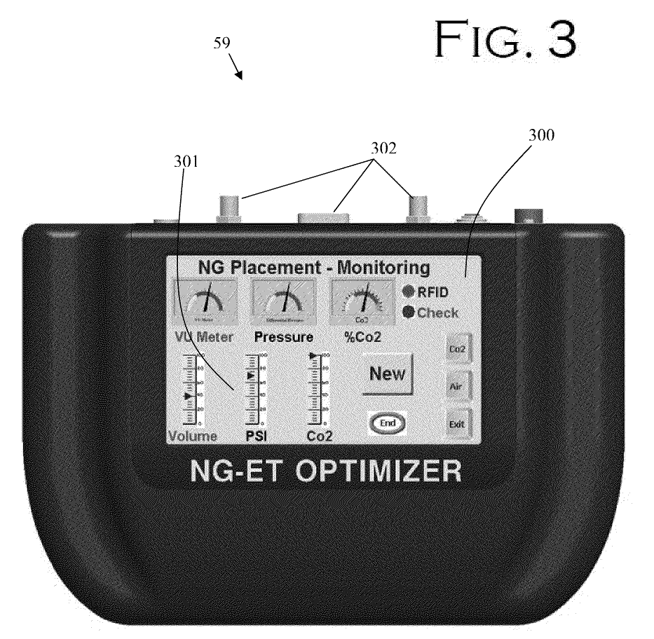

FIG. 3 illustrates one embodiment of a control panel 59 comprising a graphical user interface displaying information or data for nasogastric tube insertion and/or monitoring. Such a graphical user interface may provide a touch screen interface 300 to both display information and receive user input. The graphical user interface may assist a clinician in insertion of a medical device such as a nasogastric or endotracheal tube. Upon insertion, the graphical user interface may convert to a monitoring system for the duration of the intubation period.

FIG. 3 depicts one embodiment of a display on a graphical user interface of the present invention. The touch screen interface 300 shown provides controls of system volume for an electronic stethoscope function, wherein CO2 and pressure may easily be controlled via user touch sliders 301. Parameters may be displayed as graphical meters or indicators allowing for continual review of the patient's status. Any breach of the predetermined parameters may initiate an alarm to alert clinicians. With the RFID tag 200 monitoring the distal end of the nasogastric tube 11, a change in the parameters such as pH value may signal misplacement of the medical device. With real-time information or data being displayed on the touch screen interface 300, the control panel 59 assists clinicians equally well during either insertion or continued monitoring of a medical device, such as a nasogastric tube, within a patient. A new patient function or feature may provide for monitoring the elapsed time of the medical device placement. The alarm feature for any breached parameters may comprise several forms that may be used alone or in any combination and may include but is not limited to indicator lights, alert tones, graphical indicators, and the like. Additionally, a control panel 59 feature may allow for the purging of the nasogastric tube and may be used with the electronic stethoscope during the monitoring cycle. Redundant methods may be provided and employed to confirm proper placement and monitoring of the nasogastric tube or other medical device.

A nasogastric embodiment of the present invention, as shown in FIG. 1, may be required whenever a patient cannot provide their own means for hydration, medication, or feeding. Uses of nasogastric tubes may also provide suction to remove toxic agents from the stomach, decompress the stomach after major abdominal surgery, or remove swallowed air in patients.

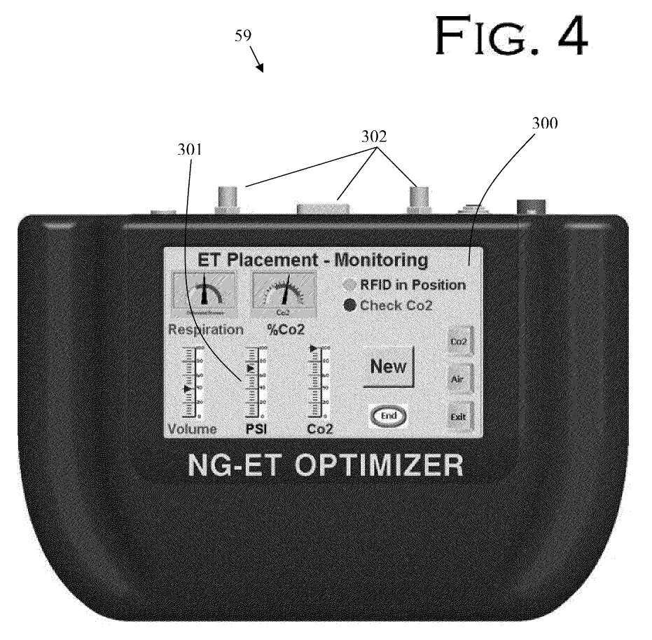

FIG. 4 depicts another embodiment of a display on a graphical user interface of the present invention. The touch screen interface 300 shown provides controls of system volume for an electronic stethoscope function, wherein CO2, pressure, and respiration rate may easily be controlled or monitored via user touch sliders 301. Such parameters may be displayed as graphical meters or indicators allowing for continual review of the patient's status. Any breach of the predetermined parameters may initiate an alarm to alert clinicians. With the RFID tag 200 monitoring the distal end of an endotracheal tube 11, a change in the parameters such as pH value may signal misplacement of the medical device. With real-time information or data being displayed on the touch screen interface 300, the control panel 59 assists clinicians equally well during either insertion or continued monitoring of a medical device, such as an endotracheal tube, within a patient. A new patient function or feature may provide for monitoring the elapsed time of the medical device placement. The alarm feature for any breached parameters may comprise several forms that may be used alone or in any combination and may include but are not limited to indicator lights, alert tones, graphical indicators, and the like. Additionally, a control panel 59 feature may allow for the purging of the endotracheal tube and may be used with the electronic stethoscope during the monitoring cycle. Redundant method may be provided and employed to confirm proper placement and monitoring of the endotracheal tube.

An endotracheal embodiment of the present invention may be required whenever a critically ill patient's air flow has been compromised and breathing assistance is indicated. Such a system may be used with patients who are ventilator or endotracheal dependent, and the present inventive system may continually monitor the CO2 gases in the patient's lungs.

The control panel 59 and/or touch screen interface 300 may provide additional screens or displays for communicating battery or power levels, wherein a low battery or power level alarm may be triggered when power drops below a specific level. Other features that may be controlled and optimized include but are not limited to back light intensity, calibration of an LCD screen, low power draw settings providing for a environmentally friendly system, and the like. As shown in FIG. 3 and FIG. 4, the control panel 59 may further comprise at least one connector 302. The at least one connector 302 may include any number of connection ports including but not limited to power supply, an RFID port, a CO2 port, an audio port, a nasogastric tube port, and the like.

As shown in FIG. 1 and FIG. 2, the feedback receiver 30 is in communication with feedback initiator 20 through a radio frequency signal. The feedback initiator 20 may comprise one component of a radio frequency identification (RFID) tag 200. In one embodiment of the nasogastric or endotracheal placement and monitoring system, the RFID tag 200 may monitor and provide pH, continuity, and temperature readings. Within the scope of the present invention, the RFID tag 200 may be secured to any medical device including but not limited to the distal end of a nasogastric or endotracheal tube of the embodiments described herein. Use of the RFID embodiment within the scope of the present invention eliminates and effectively replaces the need for a hard-wiring means between the feedback initiator 20 and the feedback receiver 30. In such a wireless embodiment, the feedback initiator 20 is in physical communication with an RF transmitter 225 and an RF antenna 220 contained within the RFID tag 200. Additionally, the feedback receiver 30 is in communication with an external RFID reader and detection system 201. In this manner, a feedback initiator 20 secured to a medical device may transmit information or data regarding the patient's immediate internal environment in real-time to the clinician via a radio frequency signal that is received and read by the feedback receiver 30 and provides for notification thereafter to a clinician. Such a wireless RFID embodiment of the present invention may be incorporated onto any known medical device and, as disclosed herein, the feedback initiator 20 may sense and monitor any number of characteristics of the environment immediately adjacent to the RFID tag 200.

In one example, the clinician uses the standard general method of placing a nasogastric tube into the proper location, which is the organ of interest for the intended procedure. Most commonly, the organ of interest is the stomach, but at times, the organ of interest may be the lungs when using an endotracheal tube to provide an airway or a suction tube to remove toxins or fluids. For example, if the stomach is the organ of interest, the distal end of nasogastric tube 11 is inserted through the nose 13 into the throat 14 and then down the esophagus 15 and into the stomach 16 of the patient. While the insertion procedure is being carried out, feedback receiver 30 receives information or data about the distal end of tube 11 from feedback initiator 20 within the RFID tag 200, and supplies that information or data to the clinician via the clinician notifying system 77, which is displayed on control panel 59 or touch screen interface 300 thereby assisting in and verifying the location of the tube 11 placement.

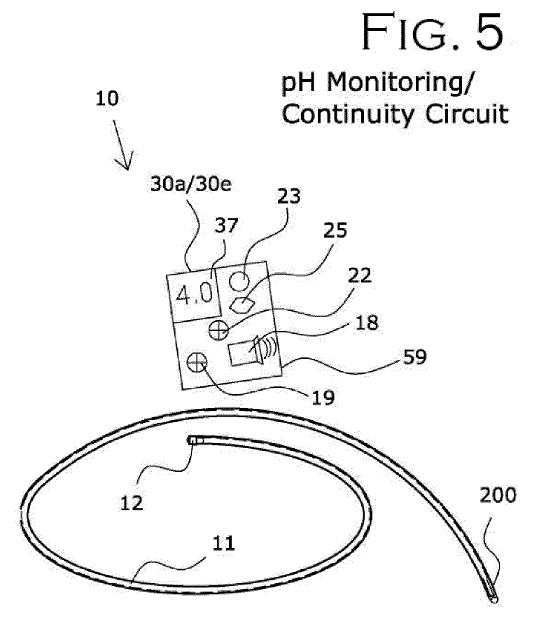

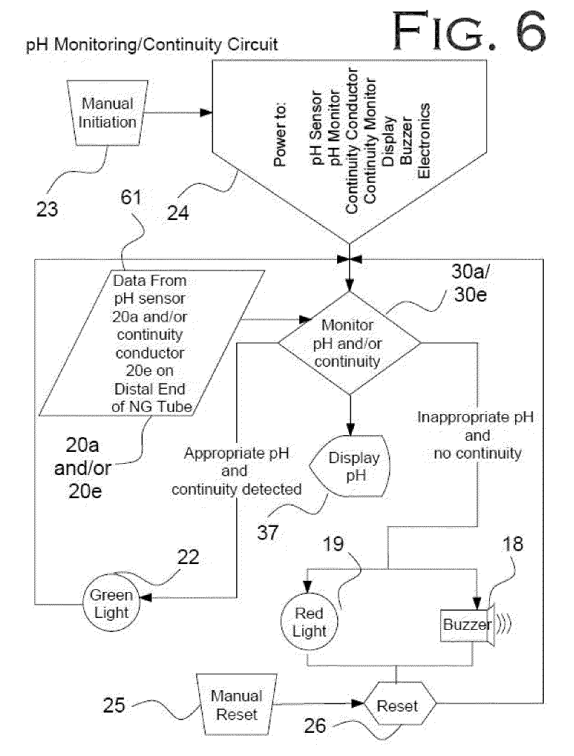

In one embodiment, as seen in FIG. 5 and FIG. 6, a pH monitoring circuit and/or continuity circuit is provided as part of the RFID tag 200. The feedback initiator 20 may comprise part of the RFID tag 200, wherein the feedback initiator 20 may comprise a pH sensor 20a and/or continuity conductor 20e. pH sensor 20a continually senses the acidity or basicity of the fluid located at the distal end of flexible tube 11 by giving a measurement of the concentration of hydrogen ions. The continuity circuit, utilizing continuity conductor 20e, is a means of determining continuity through which the conductors continually monitor the conductivity of the fluid located at the distal end of the flexible tube 11, monitoring the presence of hydrogen ions.

The continuity circuit provides a potential to continuity conductor 20e at the distal end of flexible tube 11. A radio frequency signal is used as a means to provide or supply the potential to continuity conductor 20e, and, if continuity at continuity conductor 20e is available or detected, an RF transmitter 225 (see FIG. 2) transmits the potential back to the continuity monitor 30e. Thus if conductivity is present during the presence of hydrogen ions, continuity monitor 30e is activated. The feedback receiver, continuity monitor 30e, configured with the appropriate electronics package, receives the RF output from the RF transmitter 225 that was initiated by the continuity conductor 20e, analyzes it, and outputs a reading, displayed on pH display 37 or, alternately on a separate display (such as a touch screen interface 300), on control panel 59 that can be easily read by the clinician. The pH display 37 or touch screen interface 300 serves as the notifying system that provides the clinician with the information concerning the distal end of flexible tube 11.

Although shown in FIG. 5 as providing a digital readout on pH display 37, the pH display may also be in the form of a graphical user interface or touch screen interface 300 that either displays the numerical data or a graphical indicator of the pH level (see FIG. 3 and FIG. 4). Furthermore, one or more graphical indicators or user slide bars can be used to notify the clinician of the detected information as may be indicated on the control panel 59, an LCD panel display of the graphical user interface, or a touch screen interface 300. Additionally, the detected continuity can be utilized with other circuits in the nasogastric or endotracheal tube placement and monitoring system 10 of the present invention.

Preferably an initiation button 23 is provided to manually start the power to the parts of the continuity circuit, as well as a reset button 25 to manually turn off the power.

The pH monitoring circuit may use a conventionally available pH sensor. Any pH sensor as is known in the art is within the scope of the invention, for example, combination electrode sensors or solid-state pH electrode sensors. Preferably, the pH sensor is a combination pH sensor including a pH measuring electrode that is sensitive to the hydrogen ions and develops a potential or voltage directly related to the hydrogen ion concentration of the fluid, a reference electrode that provides a stable potential against which the measuring electrode can be compared, and a preamplifier that strengthens and stabilizes the signal. The two electrodes generate a voltage related to the pH of the fluid. After conditioning in the pre-amplifier, the electrical signal communicated via radio frequency signal from a radio frequency transmitter on the RFID tag 200 to an RFID reader and detection system 201 that is in communication with feedback receiver 30. A pH sensor preferably will be chosen that has a suitable resolution of approximately 0.1 pH, that has a suitable range of pH measurements, that is suitably protected in a housing, and that is designed for use in a fluid sample environment. Preferably, to save time for the clinician at the time of intubation, the pH sensor will not require calibration before use, although all pH sensors are within the scope of the invention including those requiring calibration.

In normal operation, the RFID tag 200 remains static until it is stimulated by an external radio frequency (RF) energy source that is provided by the external RFID reader and detection system 201 (see FIG. 1).

Once the RFID tag 200 receives the RF signal, the RFID tag 200 becomes activated via the received RF signal energy and begins taking samples in “real-time” of pH, temperature, CO2 levels, and the like. It then stores the data into memory. In one example, the pH value noted for the stomach may be communicated via an RFID tag 200 mounted to the distal end of a nasogastric tube. This value information is reflected back as a reading to the microcontroller disposed within the RFID tag 200. The circuit is enabled whenever the conductivity between the two silver chloride contacts or the semi-permeable membrane 210 is within the programmed pH range. This is possible because the proper continuity is programmed into the microcontroller.

A pH monitor 30a (the feedback receiver) configured with the appropriate electronics package, receives the electrical output (via an RF signal) from pH sensor 20a, analyzes it (particularly to determine if the detected pH is appropriate for the organ of interest), and outputs a display 37 of the detected pH value that can be easily read by the clinician. The feedback receiver 30a can be configured to provide either a digital or analog readout for display 37. Furthermore, one or more indicator lights 19, 22 or graphical indicators 301 can be used to notify the clinician of the detected pH information. Additionally, the measured pH value can be utilized with other circuits in the nasogastric or endotracheal tube placement and monitoring system 10 of the present invention.

Optionally, the pH sensor 20a may communicate directly with the pH display 37 or other graphical user interface, without the benefit of the pH monitor 30a, providing a simplified variation. In this case, no analysis of the detected pH would be performed to determine if it was appropriate for the organ of interest, but the detected pH would be directly displayed the clinician in real time.

Therefore the above embodiment of the nasogastric or endotracheal tube placement and monitoring system 10 provides the pH value at a specific point in time, as well as being a permanently installed sensor for use as a long term monitoring system. A lower pH reading, such as, for example, approximately pH 5 or lower, indicates the correct placement into the stomach, while a higher pH reading, such as, for example, approximately pH 6 or greater, indicates a tube 11 inadvertently positioned in the respiratory system. If this were to occur, the clinician or caregiver would be notified by an alarm circuit of the present invention. The clinician can quickly determine the pH at the distal end of flexible tube 11 merely by looking at display 37 or the graphical user interface, instead of being required to aspirate fluid from the tube and to perform a test on it, as is currently the standard procedure. Additionally, the pH measurement will be continually displayed while the tube 11 continues to be in place, allowing the clinician to refer to it any time desired without the time investment of aspirating fluid for testing and without the time and cost investment of radiological confirmation. Furthermore, the patient's exposure to radiation is minimized as this method reduces the need for X-rays. Additionally, as shown in FIG. 3 and FIG. 4, graphical indicators 310 may be used alone or in combination with any other forms of indicators, warning lights or audible warnings.

To facilitate use of the disclosed pH monitoring system, an initiation button 23 is preferably provided to commence the power to the parts of the pH monitoring circuit, as well as a reset button 25. Also, if the pH is inappropriate for the organ of interest, a warning or indicator light 19 and a buzzer 18 are provided. For example, if the pH is 7.5 when the placement location desired for the nasogastric tube is the stomach, the preferably red indicator light 19 and the buzzer 18 are activated by the pH monitoring circuit, thereby conveniently alerting the clinician. Optionally, either the red indicator light 19 or buzzer 18 may be utilized alone as indicators. Also optionally, a preferably green indicator light 22 may be provided to allow the clinician to quickly determine that the pH is appropriate to the organ of interest, without having to read the pH meter display 37 or graphical user interface. For convenience and efficiency, preferably the initiation button 23, reset button 25, indicator light 19, indicator light 22, buzzer 18, and pH meter display 37 are incorporated into control panel 59 or the graphical user interface.

The flowchart of FIG. 6 illustrates an overview of the operation of the pH monitoring/continuity circuit. The pH monitoring/continuity circuit is started when the clinician engages a button or switch, manual initiation 23. This initiation provides power 24 to the pH sensor 20a, pH monitor 30a, continuity monitor 30e, continuity conductor 20e, pH display 37, buzzer 18 and the other electronic circuitry of the pH monitoring/continuity circuit. In block 30a/30e, the pH monitor 30a and continuity monitor 30e receive data from the pH sensor 20a and/or continuity conductor 20e (block 61) on the distal end of nasogastric tube 11, analyze the received detected pH based on the organ of interest, and determine if the detected pH is appropriate for the selected organ of interest and to activate the appropriate lights or buzzer.

In block 37, the received detected pH data is output and displayed on the pH meter display 37, other graphical user interface, or touch screen interface 300. If the pH and continuity is appropriate for the organ of interest, the indicator light 22 will be lighted (block 22). If the pH or continuity is inappropriate, the preferably red indicator light 19 will be lit and, preferably optional buzzer 18 will sound an audible alarm, which may be manually reset by the clinician by depressing reset button 25, whereby the electronics reset the circuit 26. As an alternative option, in an embodiment having a graphical user interface such as a touch screen interface 300, the indicator lights 19, 22 may be replaced or otherwise augmented with graphical indicators displayed on the interface.

When following the present inventive disclosure, any further displacement of the distal end of the medical device following its successful placement may provide a clear and recognizable change from the levels of predetermined proper continuity, regarding pH values, temperature, and the like. Real-time sampling of data including but not limited to pH values, temperature, CO2 levels, and the like may be communicated to the RFID reader and detection system 201 through a radio frequency signal transmitted by the RFID tag 200. The information may also be communicated to and recorded by the microcontroller 215. If any proper continuity reading is breeched, an alarm circuit may be activated to alert health care practitioners and such a circuit may also indicate the corrective measures that are necessary. The present invention assists not only in placement of nasogastric or endotracheal tubes but also allows for real-time monitoring of the status of the tube's distal tip. The ability to monitor live or real-time data readings from a portion of a medical device inserted within a patient allows for a previously unachieved level of safety in both patient care and medical treatment settings.

The scope of the present invention may also include variations wherein the feedback initiator 20 and feedback receiver 30 comprise temperature monitoring circuits, audio monitoring circuits, air pressure circuits, stomach placement and monitoring circuits, lung placement and monitoring circuits, or any combinations thereof within a single system of the present invention.

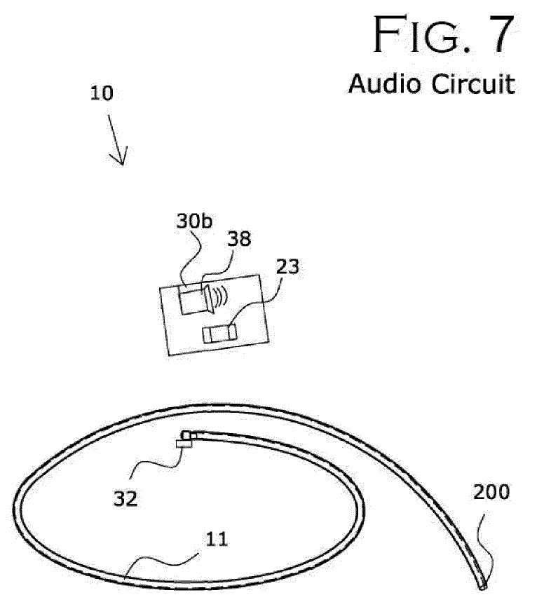

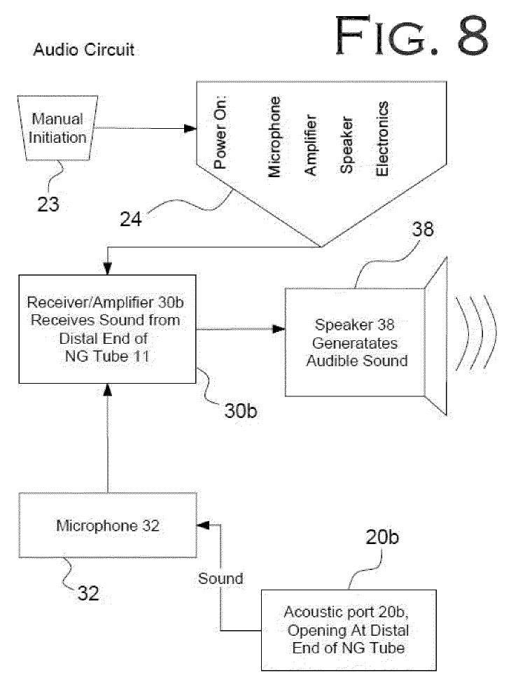

In a second embodiment, shown in FIG. 7 and FIG. 8, an audio monitoring circuit is illustrated, with the flowchart of FIG. 8 illustrating an overview of the operation of the audio monitoring circuit. The feedback initiator within the RFID tag 200 at the distal end of flexible tube 11 is an acoustic port 20b or opening using tube 11 as a means for sound to travel to the microphone 32 on the proximal end of tube 11, FIG. 7.

Feedback initiator 20 in the second embodiment preferably comprises a microphone 32 to receive the sounds from the distal end of flexible tube 11 configured with the appropriate amplifiers to amplify the received sounds and the appropriate circuitry to transmit the sounds via an RF transmitter to the speaker 38 which is configured with the appropriate circuitry to generate an audible sound that can be heard by the clinicians. The speaker 38 serves as a notifying device that provides the clinician with information concerning the distal end of flexible tube 11. As disclosed in the first embodiment, the bodily sounds may be received by the feedback initiator 20 (or microphone 32) after which an RF transmitter may send an RF signal to the feedback receiver 30b/speaker 38 to generate and audible sound for the clinician.

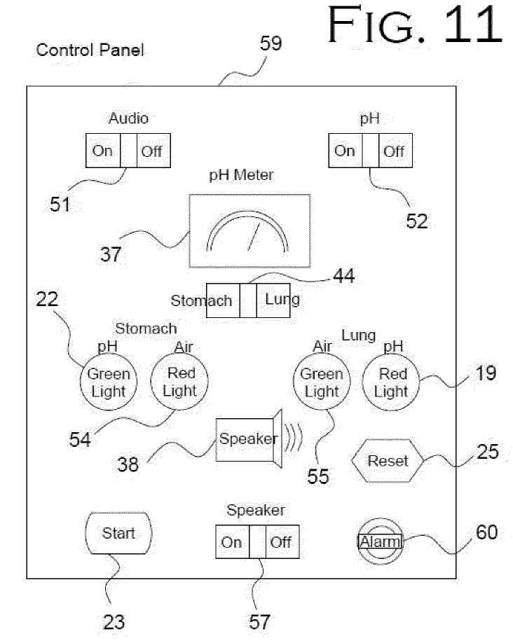

The sound reverberations and echoes from the acoustic port at the distal end of flexible tube 11, detected by microphone 32, will vary depending on the size, shape, and fluid of the cavity, thereby enabling clinicians to determine the placement location merely by listening to the audible sound from the speaker 38 and differentiating between the sounds made when a tube is correctly placed in the organ of interest, and when the tube is incorrectly located. For example, if the stomach is the organ of interest, the sounds should be typical stomach sounds, such as gurgling, whereas if the tube is incorrectly placed in the lungs, trachea, or esophagus sounds of air movement may be heard. This increases staff efficiency saving time and effort, because instead of using the traditional auscultatory method requiring the staff to insufflate air through the tube and then to use a stethoscope to listen to the sounds, the present invention provides continuous audible feedback. When the audible feedback is no longer desired, speaker 38 can be manually switched off via reset button 25 (see FIG. 11). The audio circuit may be initiated by manual initiation button 23. Radiation exposure is reduced when placement of the nasogastric tube 11 is determined by this method instead of requiring radiological confirmation.

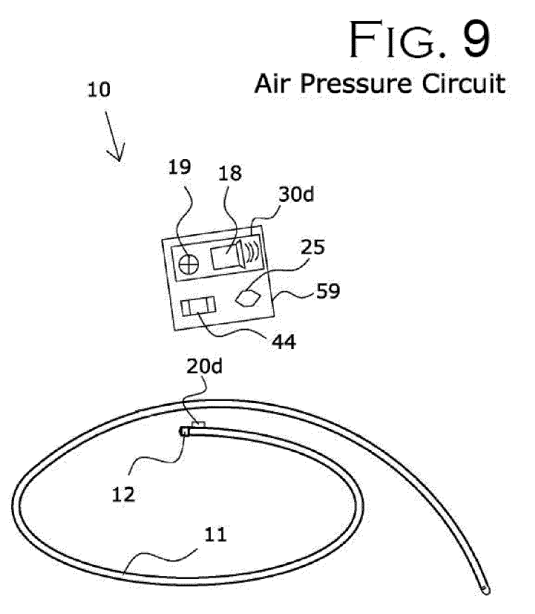

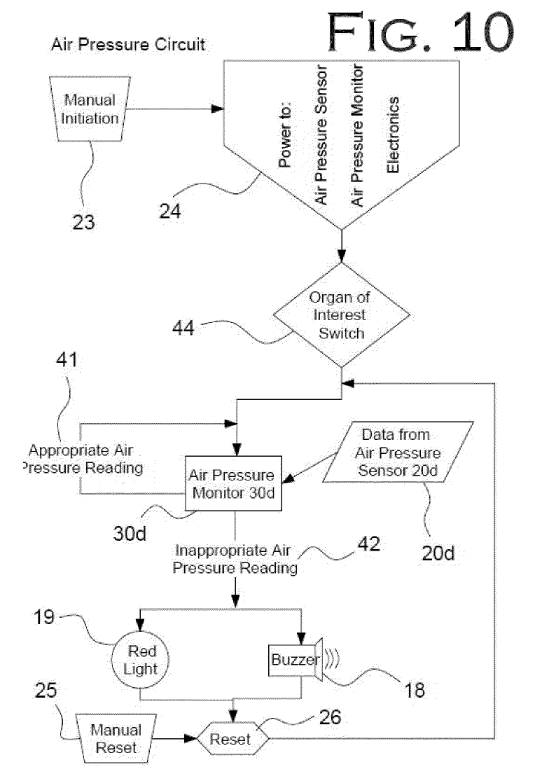

Illustrated in FIG. 9 and FIG. 10 is a third embodiment, an air pressure circuit to monitor air flow so that the attending clinician can ascertain whether the nasogastric tube is located in the airway or not, either during intubation or after intubation. The feedback initiator connected to the proximal end of flexible tube 11 is an air pressure sensor 20d. The air pressure sensor 20d is configured to measure the pressure or airflow of the air at the distal end of flexible tube 11. The detected air pressure data is transmitted to the feedback receiver, air pressure monitor 30d via an RF signal communicated from an RF transmitter at the proximal end of tube 11 to the feedback receiver, air pressure monitor 30d. Air pressure monitor 30d is configured with the appropriate electronics to receive the information from the air pressure sensor 20d, to analyze the information to determine if the information is appropriate for the organ of interest, and to send a signal to activate the notifying device, a preferably red indicator light 19 and warning buzzer 18, to alert the clinician of the air pressure or lack thereof at the distal end of flexible tube 11. In an alternative embodiment, the notifying device may comprise a graphical user interface, such as a touch screen interface 300, to provide graphical indicators to the clinician as well as provide touch screen controls (see FIG. 3 and FIG. 4).

Also preferably included in the third embodiment is an organ of interest determination switch 44 incorporated into control panel 59. The organ of interest determination switch 44 allows the attending clinician to set the circuit on either the lung or the stomach. Based on the organ of interest chosen, the circuitry and electronics are configured to display the correct reading for the chosen organ based on the data received.

A conventional air pressure sensor 20d is utilized. The air pressure sensor 20d will preferably be chosen that has a suitable air pressure range and that is space-efficient.

As illustrated in the flowchart of FIG. 10, after the manual initiation 23 of the air pressure circuit, power 24 is supplied to the various parts of the circuit, including the air pressure sensor 20d located proximally on nasogastric tube 11, feedback receiver 30d, and the other associated electronics.

Then the attending clinician manually operates the organ of interest determination switch 44 to designate the organ into which the nasogastric tube 11 will be inserted. Organ of interest determination switch 44 may optionally include a light (not shown) which will be lighted to designate the chosen organ of interest, for convenience for the clinician.

Air pressure sensor 20d then takes readings and transmits the resultant data back to the feedback receiver via an RF signal as described in association with the first embodiment. The feedback receiver, an air pressure monitor 30d, is configured with the appropriate circuitry and electronics to receive the RF signal transmitted from air pressure sensor 20d and to analyze it. If the air pressure reading is appropriate 41 for the organ of interest, the air pressure sensor 20d merely continues to monitor the air pressure.

If the air pressure reading is inappropriate 42 for the organ of interest, the feedback receiver 30d causes the notifying devices to be activated. Warning buzzer 18 emits an audible alarm and/or the warning indicator light 19 is lighted, thus producing a visual and auditory signal and notification to the clinician of the problem. These may automatically reset after a given amount of time, or alternatively, as shown, they may be manually reset when the clinician depresses reset button 25, whereby the electronics reset the circuit 26. In an embodiment utilizing a graphical user interface, the audible and visual warning indicators or graphical indicators 301 may be emitted from or displayed on the graphical user interface or touch screen interface 300 (see FIG. 3 and FIG. 4).

FIG. 11 shows a diagrammatic illustration of a control panel 59, which can be used with any of the embodiments of the present invention, but is herein illustrated as configured for a fourth embodiment of the nasogastric or endotracheal tube placement and monitoring system 10. Control panel 59 is sized and configured to conveniently integrate the circuits to be utilized, to provide easily accessible controls for the circuits, and to convey the information concerning the distal end of flexible tube 11 to the attending clinician. Control panel 59 is designed to be placed near the patient's bed or other location where the intubation could be performed. The control panel elements and the electronic components of the various embodiments should preferably be digital, although analog implementations are within the scope of the present invention.

FIG. 3 and FIG. 4 illustrate the preferred embodiment of a control panel 59 comprising a graphical user interface displaying information or data for nasogastric tube insertion and/or monitoring. Such a graphical user interface may provide a touch screen interface 300 to display information or data and receive user input. The graphical user interface may assist a clinician in insertion of a medical device such as a nasogastric or endotracheal tube. Upon insertion, the graphical user interface may convert to a monitoring system for the duration of the intubation period. All variations of switches, lights, indicators, and the like found on control panel 59 may be reproduced in digital format on a graphical user interface embodiment. A touch screen interface 300 may substitute on-screen buttons, switches, and other graphical indicators 301 for the mechanical alternatives found on alternate versions of the control panel 59. Audible alerts may be provided by speakers that may be attached on or within the graphical user interface or control panel 59.

The control panel 59 for the fourth embodiment preferably comprises the following: a start button 23 to initiate power to electronic components of the nasogastric or endotracheal tube placement and monitoring system; an organ of interest switch 44 to allow the clinician to manually designate the intended organ for the procedure; an audio on-off switch 51 to power the audio monitoring circuit; a pH monitoring/continuity circuit on-off switch 52 to power the pH monitoring/continuity circuit; a pH display 37 to display the sensed pH; a speaker 38 configured to output sounds; an on-off switch to power speaker 38; a manual reset button 25 to initiate the resetting of the circuits; an alarm 60 which displays a visible and preferably red light; a stomach air indicator preferably red light 54 to designate an inappropriate air pressure sensor reading; a stomach pH indicator preferably green indicator light 22 to indicate an appropriate pH reading; a lung air indicator light 55 to designate an appropriate air pressure sensor reading; and a lung pH indicator preferably red light 19 to indicate an inappropriate pH.

While several separate power and on-off switches are illustrated for demonstrative purposes, in practice a single on-off switch could incorporate the several circuits utilized, thus simplifying the initiation of the intubation system. Other notifying devices, such as other lights, buzzers, and alarms can additionally be incorporated to more easily allow the clinician to receive data about the distal end of flexible tube 11.

Either or both the pH and the continuity is/are monitored in block 61 using the above described pH monitoring/continuity circuit of FIG. 5 and FIG. 6. If the pH is low and/or continuity is detected 65, green indicator light 22 is lighted to signify to the clinician that the appropriate pH and/or continuity is being sensed, and the circuit continues to monitor the pH (block 61). If the pH is high and/or no continuity is detected 66 an inappropriate pH and/or continuity is being sensed.

The air pressure is monitored (block 62) by the air pressure circuit of FIG. 9 and FIG. 10. If the air pressure signifies that the tube is in an airway passage, red warning light 54 is lighted. If the pH is high and/or no continuity is detected 66 and if the air pressure signifies that the tube 11 is in an airway passage, then alarm 60 is activated with a visible red light plus, preferably, a buzzer 19. If alarm 60 is activated, the circuits can be manually reset by depressing the reset button 25, whereby the electronics reset the circuit 26. Alternatively, if desired the circuits can automatically reset after a time delay (not shown).

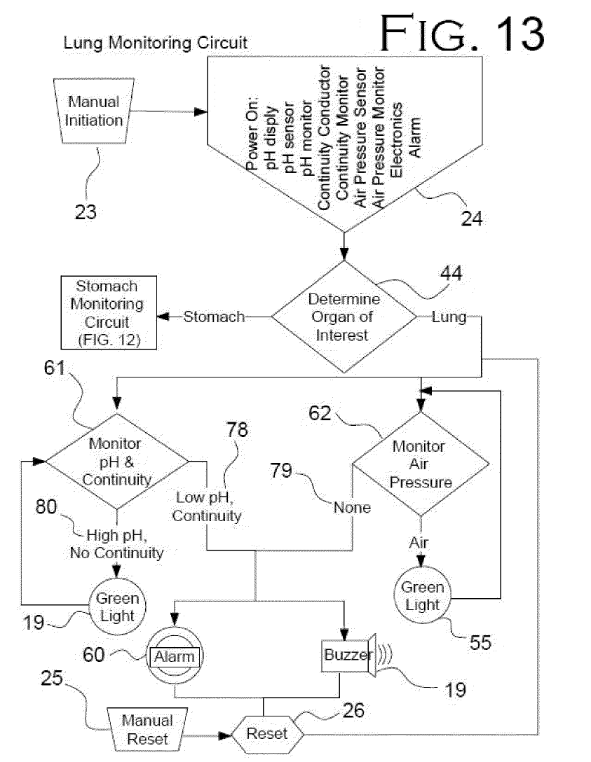

FIG. 13 illustrates a fifth embodiment, a lung placement and monitoring circuit, using the pH monitoring circuit of FIG. 5 and FIG. 6 and the air pressure circuit of FIG. 9 and FIG. 10. This circuit would be selected by manual manipulation of the organ of interest switch 44 by choosing “lung”.

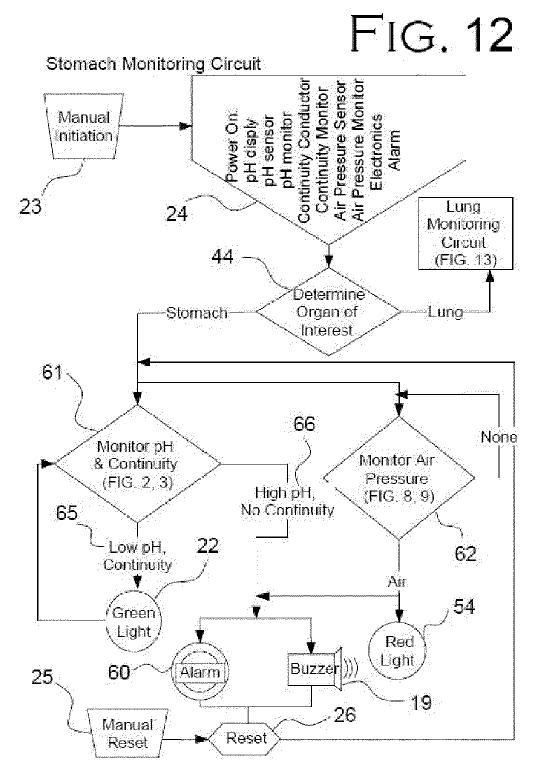

The operation of the lung placement and monitoring circuit is manually initiated by engaging the start button 23, which powers on (block 24) the air pressure sensor 20d, air pressure monitor 30d, alarm 60, pH sensor 20a, pH monitor 30a, display 37, continuity conductor 20e, continuity monitor 30e, and associated electronic components. The pH monitor 30a may be separately turned on by pH switch 52 (not shown in flowchart of FIG. 12, but illustrated in FIG. 11), or, alternatively, may be included in power on (block 24), as shown in FIG. 12.

The organ of interest, in this case the lung, may be selected by the attending clinician by manual manipulation of the organ of interest determination switch 44.

Either or both the pH and the continuity is/are monitored in block 61 using the above described pH monitoring/continuity circuit of FIG. 5 and FIG. 6. If the pH is high and/or there is no continuity 80, green indicator light 19 is lighted to signify to the clinician that the appropriate pH and/or continuity is being sensed, and the circuit continues to monitor the pH, block 61. If the pH is low and/or no continuity is detected 78, an inappropriate pH and/or continuity is being sensed.

The air pressure is monitored (block 62) by the air pressure circuit of FIG. 9 and FIG. 10. If the air pressure signifies that the tube is in an airway passage, green indicator light 55 is lighted. If the air pressure signifies that the tube is not in an airway passage and if the pH is low and/or no continuity is detected 79, alarm 60 is activated with a visible light plus, optionally a buzzer 19 may be sounded. If alarm 60 is activated, the circuits can be reset 26 by manually operating button 25. Alternatively, if desired, the circuits can automatically reset after a time delay (not shown).

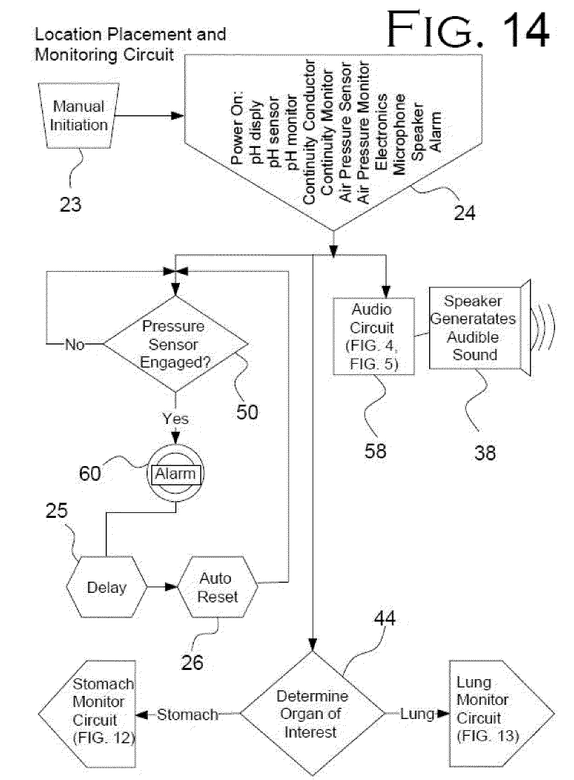

FIG. 14 illustrates combined circuits to form a nasogastric tube location placement and monitoring circuit, a sixth embodiment, utilizing the pressure circuit of FIG. 9 and FIG. 10, the audio monitoring circuit of FIG. 7 and FIG. 8, stomach placement and monitoring circuit of FIG. 12 (which utilizes the pH sensor/continuity circuit of FIG. 5 and FIG. 6 and the air pressure circuit of FIG. 9 and FIG. 10) and the lung placement and monitoring circuit of FIG. 13 (which utilizes the pH sensor/continuity circuit of FIG. 5 and FIG. 6 and the air pressure circuit of FIG. 9 and FIG. 10).

Manual initiation 23 powers the air pressure sensor 20d, air pressure monitor 30d, pH sensor 20a, pH monitor 30a, display 37, continuity conductor 20e, continuity monitor 30e, microphone 32, speaker 38, alarm 60, and associated electronic components. Some components can be optionally be separated powered on, such as, for example, pH switch 52 can be used to turn on the pH sensor 20a, pH monitor 30a, continuity conductor 20e, continuity monitor 30e, and pH display 37; the speaker switch 57 can turn on the speaker 38; and the audio monitoring circuit can be powered by audio switch 51.

Then the organ of interest is selected by the attending clinician by choosing on the interface the appropriate function, in this embodiment either a nasogastric tube for the stomach or an endotracheal tube for the lungs, indicated by the organ of interest determination switch 44.

The pressure sensor circuit 50 determines if the pressure sensor 20c is engaged, alerting the clinician to a problem via alarm 60, as described in FIG. 9 and FIG. 10.

The audio circuit 58 generates audible sounds via speaker 38, as described in FIG. 7 and FIG. 8.

After the determination of the organ of interest 44, either the stomach placement and monitoring circuit of FIG. 12 or the lung placement and monitoring circuit of FIG. 13 is activated. Both the stomach placement and monitoring circuit and the lung placement and monitoring circuit utilize the air pressure circuit and the pH monitoring/continuity circuit.

The pH and/or continuity is monitored and displayed using the pH monitoring/continuity circuit, with the clinician alerted to an appropriate pH and continuity or to an inappropriate pH and continuity, as described in FIG. 5 and FIG. 6.

The air pressure is monitored via the air pressure sensor 20d using the air pressure circuit as described in FIG. 9 and FIG. 10, with the clinician alerted to an appropriate amount of sensed air or to an inappropriate amount of sensed air.

As with the above embodiments, if alarm 60 is activated, the circuits can be manually reset by depressing the reset button 25, whereby the electronics reset the circuit 26. Alternatively, if desired the circuits can automatically reset after a time delay (not shown).

While a limited number of combinations of the provided circuits have been illustrated, additionally, many other combinations of two or more of the provided circuits can be utilized to provide information to the clinician about the distal end of the tube 11.

Thus by the utilization of one or a combination of the provided circuits, the information about the location of the distal end of the tube 11 is monitored during intubation and as long as the tube is inserted. If the distal end moves out of the organ of interest, alarm 60 lights up and, preferably, emits an audible warning sound, so corrective measures can be taken by the attending staff member. The provided nasogastric or endotracheal tube placement and monitoring system eliminates the need to use the “blind” placement techniques currently in use and thus minimizes staff errors.

From the foregoing, it will be apparent that the nasogastric or endotracheal tube placement and monitoring system 10 of the current invention provides an efficient system that assists both in the intubation and in the long-term monitoring of the tube, which can be utilized in a variety of organs or bodily spaces including but not limited to the stomach and the lungs. The nasogastric or endotracheal tube placement and monitoring system 10 of the present invention improves staff efficiency while reducing the patient's exposure to radiation as well as reducing the cost, by providing a system that confirms the placement location of the nasogastric tube or endotracheal tube without radiological confirmation.

Since many modifications, variations, and changes in detail can be made to the described preferred embodiments of the invention, it is intended that all matters in the foregoing description and shown in the accompanying figures be interpreted as illustrative and not in a limiting sense. Thus, the scope of the invention should be determined by the appended claims and their legal equivalents, and not by the examples given.

What is claimed is:

1. A placement and monitoring system for use by a clinician during insertion of a medical device within a patient, comprising:
a radio frequency identification tag secured to said medical device, said radio frequency identification tag comprising at least one feedback initiator configured to provide information concerning the location of said radio frequency identification tag within said patient;
at least one feedback receiver configured to receive said information from said at least one feedback initiator; and
at least one notifying device configured to present said information concerning the location of said radio frequency identification tag to said clinician;
wherein said information concerning the location of said radio frequency identification tag within said patient is communicated from said at least one feedback initiator to said at least one feedback receiver via transmission of a radio frequency signal.

2. The placement and monitoring system of claim 1, wherein said medical device comprises a nasogastric tube and said radio frequency identification tag is secured to a distal end of said nasogastric tube.

3. The placement and monitoring system of claim 1, wherein said medical device comprises an endotracheal tube and said radio frequency identification tag is secured to a distal end of said endotracheal tube.

4. The placement and monitoring system of claim 1, wherein said medical device comprises a catheter and said radio frequency identification tag is secured to a distal end of said catheter.

5. The placement and monitoring system of claim 1, wherein transmission of said radio frequency signal occurs between said at least one feedback initiator in communication with a radio frequency transmitter and said at least one feedback receiver in communication with a radio frequency identification reader and detection system.

6. The placement and monitoring system of claim 1, wherein said radio frequency identification tag is disposed within a plastic capsule.

7. The placement and monitoring system of claim 6, wherein at least two silver chloride contacts are mounted on said plastic capsule and the transmission of said radio frequency signal is enabled when said radio frequency identification tag is disposed in a conductive media.

8. The placement and monitoring system of claim 1, wherein said radio frequency identification tag further comprises an antenna, wherein said antenna provides the greatest signal strength when measured along the central axis of said antenna.

9. The placement and monitoring system of claim 1, wherein said at least one feedback initiator comprises a pH sensor, wherein said at least one feedback receiver comprises a pH monitor, and wherein said at least one notifying device comprises a pH display.

10. The placement and monitoring system of claim 1, wherein said at least one feedback initiator comprises a continuity conductor and said at least one feedback receiver comprises a continuity monitor.

11. The placement and monitoring system of claim 1, wherein said at least one feedback initiator comprises a temperature sensor, wherein said at least one feedback receiver comprises a temperature monitor.

12. The placement and monitoring system of claim 1, wherein said at least one feedback initiator comprises an acoustic port configured to transport sound from a distal end of said medical device to a proximal end of said medical device, wherein said at least one feedback receiver comprises a microphone, and wherein said at least one notifying device comprises a speaker.

13. The placement and monitoring system of claim 1, wherein said at least one feedback initiator comprises an air pressure sensor, wherein said at least one feedback receiver comprises an air pressure monitor, and wherein said at least one notifying device comprises at least one graphical indicator.

14. A placement and monitoring system for use by a clinician during insertion of a medical device within a patient, comprising:
a radio frequency identification tag secured to said medical device, said radio frequency identification tag comprising a pH sensor electrically connected to a radio frequency transmitter, wherein said pH sensor detects the pH of a fluid immediately adjacent said radio frequency identification tag and outputs a detected pH signal to said radio frequency transmitter;
a pH monitor for receiving said detected pH signal from said radio frequency transmitter; and
a pH display in electrical communication with said pH monitor, said pH display receives said detected pH signal and displays said detected pH signal to said clinician.

15. The placement and monitoring system of claim 14, wherein said medical device comprises a nasogastric tube and said radio frequency identification tag is secured to a distal end of said nasogastric tube.

16. The placement and monitoring system of claim 14, wherein said medical device comprises an endotracheal tube and said radio frequency identification tag is secured to a distal end of said endotracheal tube.

17. The placement and monitoring system of claim 14, wherein said medical device comprises a catheter and said radio frequency identification tag is secured to a distal end of said catheter.

18. The placement and monitoring system of claim 14, further comprising:
a control panel comprising an enclosure for electronics, wherein said pH monitor and said pH display are disposed within said control panel for facilitating use by said clinician.

19. A placement and monitoring system for use by a clinician during insertion of a medical device within a patient, comprising:
a radio frequency identification tag secured to said medical device, said radio frequency identification tag comprising:
a pH sensor for detecting the pH of a fluid adjacent said pH sensor, wherein said pH sensor outputs a detected pH signal;
a radio frequency transmitter in electrical communication with said pH sensor, wherein said radio frequency transmitter receives said detected pH signal from said pH sensor;
an antenna in electrical communication with said radio frequency transmitter, wherein said antenna transmits said detected pH signal; and
a capsule enclosing said radio frequency identification tag and providing for biocompatibility of said radio frequency identification tag with said patient; and
a control panel comprising an enclosure for electronics disposed external to said patient, said control panel further comprising:
a pH monitor for receiving said detected pH signal transmitted from said antenna; and
a pH display in electrical communication with said pH monitor, wherein said pH display receives said detected pH signal and displays said detected pH signal to said clinician;
wherein said radio frequency identification tag is disposed on the distal end of said medical device and said medical device is selected from the group consisting of a nasogastric tube, an endotracheal tube, and a catheter.

20. The placement and monitoring system of claim 19, wherein said pH display comprises a graphical user interface further comprising a touch screen interface for both receiving input from said clinician and displaying said detected pH signal to said clinician.
